(19) United States
(12) Patent Application Publication
(10) Pub. No.: US 2012/0191175 A1
(43) Pub. Date: Jul. 26, 2012

(54) Tubular Stent with Rotatable Connections and Method of Making

(75) Inventors: Michael Costa, Santa Rosa, CA (US); Lance Ensign, Santa Rosa, CA (US); John Richardson, South Jordan, UT (US); Justin Goshgarian, Santa Rosa, CA (US); Mark Hoff, Windsor, CA (US); Rui Lam, Santa Rosa, CA (US); John Imschweiler, Santa Rosa, CA (US)

(73) Assignee: Medtronic Vascular, Inc., Santa Rosa, CA (US)

(21) Appl. No.: 13/010,599

(22) Filed: Jan. 20, 2011

(51) Int. Cl. A61F 2/82; B21F 45/06

(52) U.S. Cl. 623/1.15; 140/71 R

(57) ABSTRACT

A tubular stent includes a plurality of bands aligned generally along a common longitudinal axis. A first band has a plurality of first crowns and a second band adjacent to the first band has a plurality of second crowns. A connecting ring connects the first band to the second band, wherein one of the first crowns is rotatably disposed through a lumen of the connecting ring and one of the second crowns is fused to the wall of the connecting ring outside of the lumen of the connecting ring. Methods of making tubular stents with rotatable connections are also disclosed.

### FIELD OF THE INVENTION

The present invention is directed to intraluminal stents for use in maintaining open vessel lumens, the intraluminal stent having flexible connections between adjacent bands for improved flexibility for tracking around bends of vessels. The present invention is further directed to methods of making stents with flexible connections.

### BACKGROUND OF THE INVENTION

A wide range of medical treatments have been previously developed using “endoluminal prostheses,” which terms are herein intended to mean generally tubular medical devices adapted for temporary or permanent implantation within body lumens, including both naturally occurring or artificially made lumens. Examples of lumens in which endoluminal prostheses may be implanted include, without limitation: arteries, such as those located within the coronary, mesentery, peripheral or cerebral arteries or veins; gastrointestinal tract; biliary tract; urethra; trachea; hepatic shunts; and fallopian tubes. Various types of endoluminal prostheses have also been developed, each providing a uniquely beneficial structure to modify the mechanics of the targeted luminal wall.

For example, stent prostheses have been previously disclosed for implantation within body lumens. Various stent designs have been previously disclosed for providing artificial radial support to the wall tissue, which forms the various lumens within the body, and often more specifically within the blood vessels of the body.

Cardiovascular disease, including atherosclerosis, is the leading cause of death in the U.S. The medical community has developed a number of methods and devices for treating coronary heart disease, some of which are specifically designed to treat the complications resulting from atherosclerosis and other forms of coronary arterial narrowing, which are generally termed stenoses. One method for treating arterial stenoses is percutaneous transluminal coronary angioplasty, commonly referred to as “angioplasty,” “PTA” or “PTCA.” The objective in balloon angioplasty is to enlarge the lumen in the affected segment of the coronary artery by radial hydraulic expansion. The procedure is accomplished by inflating a balloon of a balloon catheter within the narrowed lumen of the coronary artery. In some instances the vessel restenoses chronically, or closes down acutely, negating the positive effects of the angioplasty procedure.

To provide radial support to the treated vessel in order to prolong the positive effects of PTCA, a stent may be implanted in conjunction with the procedure. Effectively, the stent overcomes the tendency of the expanded vessel walls of some patients to close back down, thereby maintaining a more normal flow of blood through that vessel than would be possible if the stent were not in place. Under this procedure, the stent may be collapsed to an insertion diameter and mounted on or within a stent delivery catheter. The delivery catheter is inserted into a body lumen at a site remote from the diseased vessel and is navigated through the often tortuous vascular system so that the stent may be delivered to the desired site of treatment and expanded to its desired diameter for implantation against the vessel wall.

Access to a treatment site is most often reached by first entering the femoral artery using the common Seldinger technique. Then, a flexible guiding catheter is inserted through a sheath into the femoral artery. The guiding catheter is advanced through the femoral artery into the iliac artery and into the descending aorta. Further advancement of the guiding catheter involves passing the catheter distal end through the aortic arch into the ascending aorta where entry may be gained to either the left or the right coronary artery, as desired. To reach the stent implantation site, the stent delivery catheter must be directed through the guiding catheter and then through potentially tortuous and small caliber conduits of the body lumen. Therefore, the stent must be capable of being reduced to a small insertion diameter and must be flexible.

Stents come in a variety of shapes and sizes. For example, helical stents may be formed by bending a wire into a waveform and helically winding or wrapping the waveform into the tubular shape of the stent. An example of a helical wound stent can be found in U.S. Pat. No. 4,886,062 to Wiktor, the disclosure of which is incorporated herein by reference in its entirety. Adjacent helical windings of a helically wound stent may be connected together.

Stents may also be made by connecting sinusoidally shaped cylindrical elements together along a common longitudinal axis to form a tube. The cylindrical elements may be welded together at apices or crowns of adjacent segments, utilizing connecting elements, or other connecting mechanisms. The shape of the sinusoidal cylindrical elements is described, for example, in U.S. Pat. No. 6,344,053 to Boneau, the disclosure of which is incorporated herein by reference in its entirety.

In another example, U.S. Pat. No. 6,565,599 to Hong et al., the disclosure of which is incorporated herein by reference in its entirety, describes cylindrical elements formed from sinusoidally shaped segments which are interconnected by elongated struts of a flexible polymer material, which hold the cylindrical elements apart from one another. U.S. Pat. No. 6,475,237 to Drasler et al., the disclosure of which is incorporated herein by reference in its entirety, describes a strut wherein a portion thereof is made thinner and more flexible such that the strut can flex at those locations.

U.S. Pat. No. 5,035,706 to Gianturco, the disclosure of which is incorporated herein by reference in its entirety, describes the use of interlocking rings to connect adjacent cylindrical elements. U.S. Pat. No. 6,387,122 to Cragg, the disclosure of which is incorporated herein by reference in its entirety, describes a helical stent in which subsequent windings are connected by loop members made from sutures, staples or rings of metal or plastic.

The different types of connecting elements discussed above for connecting adjacent cylindrical segments or windings may require a compromise between stent coverage or scaffolding by the stent at the treatment site when the stent is deployed, flexibility of the stent during delivery to and implantation of the stent at the treatment site, and/or security of the connection between adjacent cylindrical elements or helical windings of the stent. Elongated axial connecting elements, for example, may provide increased flexibility over having cylindrical segments that are welded directly to each other. However, elongated axial connecting elements may separate the segments, providing less scaffolding by the stent at the treatment site. It is desirable to maximize flexibility, scaffolding and security of the connection between bands of a stent.

### BRIEF SUMMARY OF THE INVENTION

An intraluminal stent device includes at least two bands with a ring connecting the bands together. In one embodiment, a stent includes a plurality of bands aligned generally along a common longitudinal axis. The plurality of bands includes at least a first band having a plurality of first crowns and a second band adjacent to the first band and having a plurality of second crowns. The wire of the first band is rotatably disposed through a lumen of the ring, and the exterior surface of the ring is fused to the wire of the second band.

In an embodiment of a method of making a stent, a plurality of connecting rings are located along a wire such that the wire is disposed through the lumens of the connecting rings. The wire is formed into a zigzag waveform. The waveform is helically wound into a generally tubular configuration having a plurality of helical windings such that a crown of the first helical winding is rotatably disposed through a lumen of one of the connecting rings and a crown of the second adjacent helical winding is disposed adjacent a wall of the connecting ring and outside of the ring lumen. The wall of the connecting ring is then fused to the crown of the second helical winding to form a rotatable connection between one or more crowns of the first and second helical windings.

In another embodiment of a method of making a stent, the connecting rings are temporarily mounted on a cylindrical mandrel. The connecting rings are generally C-shaped such that a wall of the connecting ring includes an open longitudinal slot. A wire is formed into a zigzag waveform. The waveform is helically wrapped around the mandrel into a generally tubular configuration having a plurality of helical windings and a crown of the first helical winding is slipped or forced through a slot into the lumen of one of the connecting rings. A crown of the second helical winding is disposed adjacent and outside the wall of the connecting ring. The wall of the connecting ring is fused to the wire of the second helical winding to form a rotatable connection between the first helical winding and the second helical winding.

In another embodiment of a method of making a stent a first cylindrical element and a second cylindrical element are aligned adjacent to each other along a common longitudinal axis, wherein the first cylindrical element and the second cylindrical element each include a wire in a zigzag configuration. A connecting ring is rotatably disposed about a crown of the first cylindrical element and a crown of the second cylindrical element is disposed adjacent and outside a wall of the connecting ring. The wall of the connecting ring is fused to the crown of the second cylindrical element to rotatably connect the first cylindrical element to the second cylindrical element.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of an embodiment of a stent in accordance with the invention.

FIG. 2 is a plan view of a portion of the stent of FIG. 1 cut open and laid flat.

FIG. 3 is an enlarged side view of a connection between crowns of adjacent helical windings of the stent of FIG. 1.

FIG. 4 is a cross-sectional view along line 4-4 of FIG. 3.

FIG. 5 is a schematic perspective view of an embodiment of a ring for connecting adjacent bands of a stent.

FIG. 6 is a schematic perspective view of another embodiment of a ring for connecting adjacent bands of a stent.

FIGS. 7-9 illustrate an embodiment of a method of making the stent of FIG. 1.

FIGS. 10-16 illustrate an embodiment of a method for making the stents of FIGS. 1 and 26 with a plurality of the rings of FIG. 6.

FIGS. 17-22 illustrate another embodiment and method making the stent of FIG. 1.

FIGS. 23-25 illustrate another embodiment and method making the stent of FIG. 1.

FIG. 26 is a side view of another embodiment of a stent in accordance with the invention.

FIGS. 27-31 illustrate a method making the stent of FIG. 26.

FIG. 32 illustrates a separable crown-to-crown connector in another embodiment of the stent of FIG. 26.

### DETAILED DESCRIPTION OF THE INVENTION

Specific embodiments of the present invention are now described with reference to the figures, where like reference numbers indicate identical or functionally similar elements. As noted above, tubular stents come in two general configurations: helically wound stents and stents with cylindrical elements connected together. As used herein, the term “bands” may refer generally to helical windings of a helically wound stent or cylindrically shaped segments, rows, columns, or other similar items or terms known to those of ordinary skill in the stent art.

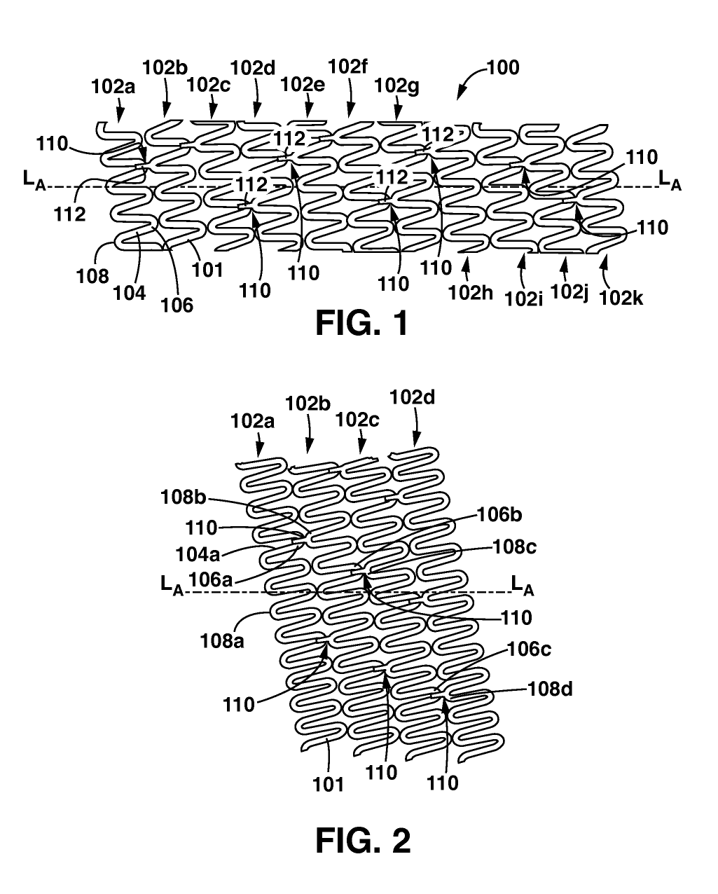

FIG. 1 shows a stent 100 made from a wire 101 bent into a waveform and helically wrapped around a longitudinal axis L_{A} to form the stent 100. The term “wire” as used herein means any elongated wire or filament or group of wires or filaments. The wire may be made from any biocompatible material used to form a stent such as stainless steel, nickel-cobalt-chromium-molybdenum “superalloy,” titanium-nickel (nitinol) or any other biologically compatible shape-memory material, magnesium, iridium, titanium, tantalum, gold, copper or copper alloys, steel alloys containing chromium, cobalt, tungsten, combinations of these materials, and/or composite layers of any of the materials listed. Further, several distinct wires may be twisted together or butt-welded in order to form a continuous wire, or a drawn-filled-tube (DFT) wire may be used wherein a core of a first material is surrounded by a second material. Stents in accordance with embodiments hereof may be either balloon expandable or self-expanding depending on the material selected for the wire used to form the waveform and or subsequent processing steps utilized during stent formation as would be understood by one of ordinary skill in the art of stent design.

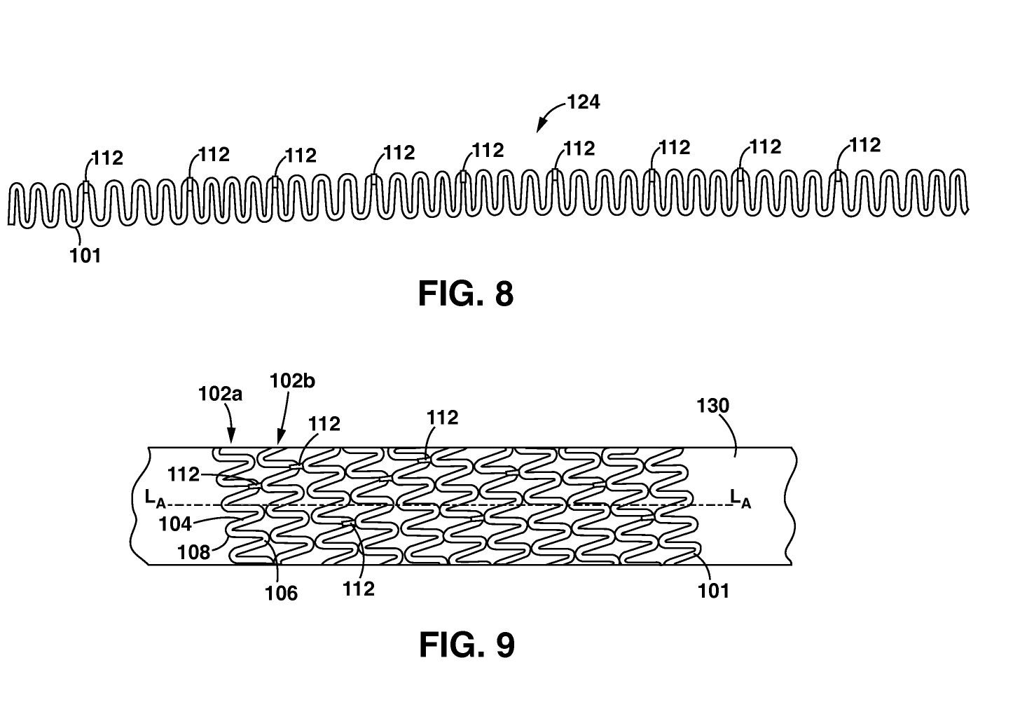

As shown in FIGS. 1 and 8, the pattern of waveform 124 can be described as meandering, zigzag or generally sinusoidal including generally straight segments or struts 104 interconnected by crowns 106, 108. Crowns 106, 108 are arbitrarily identified herein for convenience of illustration e.g. in FIGS. 1 and 2 such that crowns opening to the right are crowns 108 and crowns opening to the left are crowns 106. Adjacent helical windings or bands 102a-102k are connected together at connections 110. Connections 110 may occur between every pair of adjacent crowns 106, 108 or only some of the adjacent crowns 106, 108, as shown in FIG. 1. For example, and not by way of limitation, FIG. 2 shows bands 102a-102d of stent 100 cut open and laid flat and wherein connections 110 are located between every fourth adjacent pair of crowns 106, 108. In other words, proceeding cylindrically around the stent 100, after a connection 110 between crowns 106, 108 of adjacent bands 102, there are three pairs of adjacent crowns 106, 108 that are not connected together. Those of ordinary skill in the art of stent design would recognize that the connections 110 may be spaced closer together or farther apart, and that the spacing need not be uniform along the length of the stent.

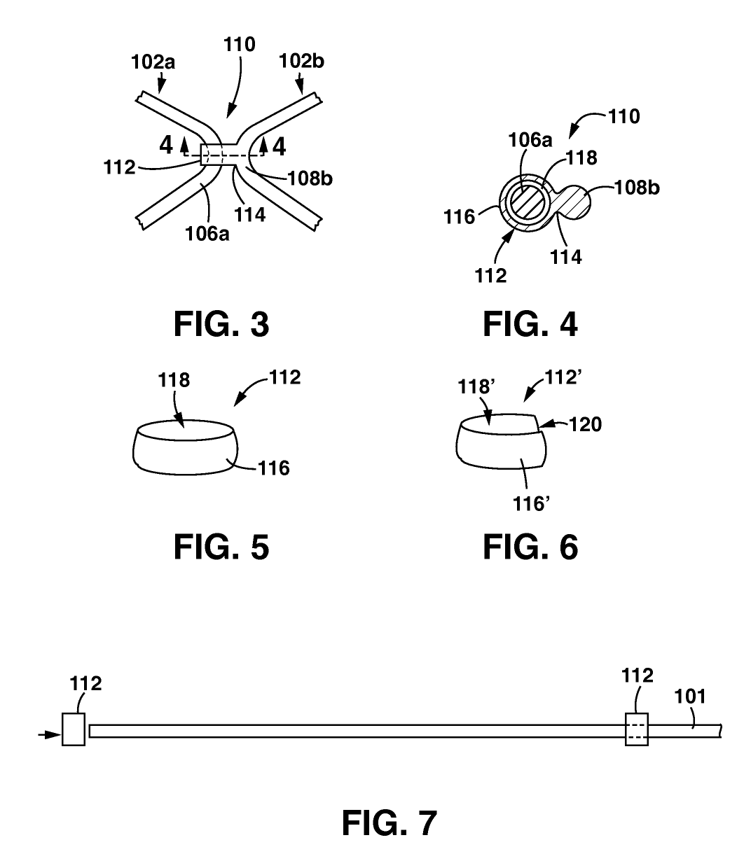

FIGS. 3 and 4 show enlarged views of one of the connections 110 between band 102a and 102b of stent 100. Connection 110 is formed by a short tube or ring 112 that surrounds crown 106a and is fused to crown 108b at fusion 114. Thus, wire 101 at crown 106a extends rotatably through a lumen 118 of ring 112 and an outside surface of a wall 116 of ring 112 is affixed to crown 108a. Those of ordinary skill in the art would recognize that ring 112 may be fused to either of the crowns and looped around the other crown. Further, some of the connections 110 may have the fusion 114 on one side and other connections 110 may have the fusion 114 on the other side. Fusion 114 may be accomplished by welding, laser welding, soldering, brazing or other methods that permanently attach ring 112 to crown 108b.

FIG. 5 shows an example of a ring 112 that can be used for connection 110. As explained above, ring 112 is in the form of a short cylindrical tube or loop including a lumen 118 formed by a wall 116. Ring 112 may be made from the same materials described above as being commonly used for wire to form a stent. FIG. 6 shows another example of a ring 112′ that is similar to ring 112 except that ring 112′ is not a closed ring. Instead, ring 112′ includes an open slot 120 extending the full length of wall 116′ such that ring 112′ is substantially C-shaped or U-shaped in end view or transverse cross-section. Slot 120 forms a pair of opposing edges where it intersects the exterior surface of ring 112′. Ring 112′ may be used interchangeably with ring 112, and may be useful in some embodiments for forming a stent where ring 112 may not be used effectively, as will be explained in more detail below.

Connections 110 provide enhanced bending flexibility to stent 100 because the crowns that are disposed in the lumens 118 of rings 112 may rotate freely with respect to the crown of the adjacent band to which the ring 112 is affixed. As compared to stents having welded connections between adjacent crowns wherein bending the stent imparts bending strain to all the elements associated with the connection, connection 110 is a substantially frictionless hinge joint that sustains negligible strain when the stent is flexed. Thus, navigating a catheter carrying stent 100 through tight-radius bends in a patient's vasculature is expected to require less translational force than navigating a catheter carrying the same stent having rigid crown-to-crown connections such as weld joints.

FIGS. 7-9 illustrate an embodiment of a method of making a stent 100 with connections 110. As shown in FIG. 7, a straight portion of a wire 101 is provided. Rings 112 are threaded onto wire 101 such that wire 101 is disposed through lumen 118 of each ring 112. Rings 112 are advanced to the selected locations along wire 112 such that after further processing, a crown of one band will be disposed through lumen 118 of ring 112 and a crown of an adjacent band will be disposed adjacent to an exterior or outer surface of wall 116 of ring 112. During further processing, rings 112 may be temporarily held at the selected locations by a small amount of adhesive, swaging, press-fit, or other methods known to those skilled the art. For example, and not by way of limitation, a small amount of adhesive may be applied to wire 101 at the desired locations for rings 112. This adhesive may hold rings 112 in place until wire 101 is formed into waveform 124 or until the rings 112 are fused as described below. The adhesive may be removed by a solvent or other methods known to those skilled in the art to ensure that each ring 112 rotates freely about wire 101. With the rings 112 temporarily fixed in their selected locations along straight wire 101, the wire may then be bent into a waveform 124, as shown in FIG. 8. The wire 101 may be bent into the waveform 124 by bending the wire 101 around posts that are laid out in a fixture (not shown) to create the desired waveform pattern. Alternatively, the wire 101 may be bent into the desired waveform 124 utilizing “fingers” of a machine to bend the wire into the desired pattern, for example, as described in U.S. patent application Ser. No. 12/428,581, filed Apr. 23, 2009, which is incorporated by reference herein in its entirety. Other methods may also be used to bend the wire into the desired pattern, such as those described in U.S. Provisional Application No. 61/244,049, filed Sep. 20, 2009, which is incorporated by reference herein, and other methods known to those of ordinary skill in the art. As shown in FIG. 8, rings 112 are located at the desired crown locations along wire 101. In this embodiment, rings 112 are located every fourth crown facing a certain direction. Those of ordinary skill in the art of stent design would recognize that the number and location of rings 112 may be varied in order to provide the desired flexibility and connecting properties for the stent 100.

With the rings 112 located at the selected locations on waveform 124, the waveform then is helically wound into a generally tubular configuration. As illustrated in FIG. 9, waveform 124 may be helically wrapped around a mandrel 130, which may be a solid cylinder or a hollow tube. With waveform 124 helically wrapped around mandrel 130, the outer surface of wall 116 of each ring 112 abuts crown 108b and is permanently affixed thereto by fusion joint 114, which may be formed by welding, soldering, or other methods known to those of ordinary skill in the art. The fusing step results in connection 110 of stent 100 as shown in FIGS. 1-4, with crown 106a of one helical band rotatably disposed through lumen 118 of ring 112 and crown 108b of the adjacent helical band fused with the exterior surface of wall 116 of the ring 112. In an alternative method, the step of loading rings 112 onto wire 101 to the desired crown locations may be performed after wire 101 is bent into the desired waveform 124, instead of prior to the bending step as described above.

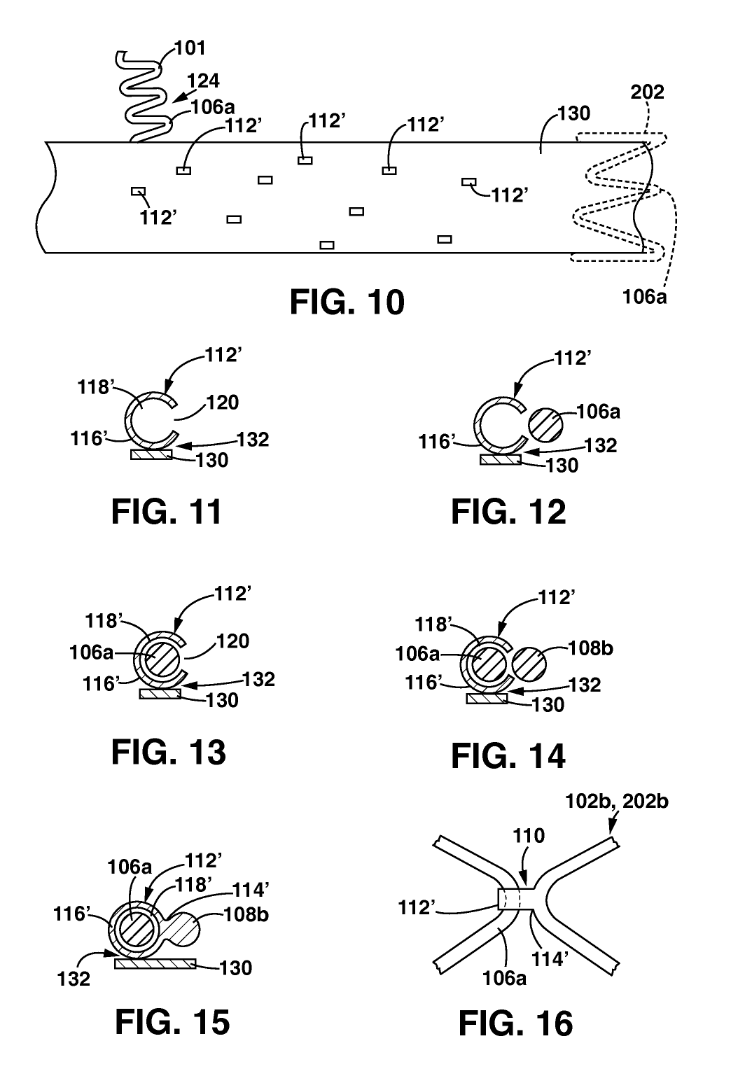

Another method of making stent 100 and also stent 200 is shown in FIGS. 10-16. In this embodiment, rings 112′ are not loaded onto wire 101 by threading an end of wire 101 through lumens 118′. Instead, wire 101 without closed rings 112 is bent into a waveform, as shown, for example, in FIG. 8. In the alternative method, using generally C-shaped rings 112′ instead of closed rings 112 may simplify placing the rings at the desired crown locations after wire 101 has been bent into waveform 124 because a selected portion of waveform 124 may be slid or snapped transversely through slot 120 into lumen 118′ of each ring 112′. Generally C-shaped rings 112′ may be temporarily affixed to an outer surface of mandrel 130 at appropriate locations to receive selected crowns 106a as waveform 124 is helically wrapped around mandrel 130, as shown in FIG. 10. Rings 112′ may be temporarily affixed to mandrel 130 by an adhesive 132, or any other attachment means that can be selectively removed as described below. FIG. 11 shows a cross-section through a portion of mandrel 130 where ring 112′ is located. Those of ordinary skill in the art would recognize that the orientation of ring 112′ on mandrel 130 may be varied depending on which crowns 106, 108 are to be received by ring 112′, or the shape of waveform 124, or the manner in which waveform 124 is wrapped around mandrel 130. For example, in the embodiment illustrated in FIGS. 11-16, slot 120 faces to the right, resulting in a connection 110 wherein crown 106a is rotatably disposed within ring 112′ and crown 108b is fused to wall 116′ blocking slot 120 so as to substantially close the slot. Alternatively (not shown), slot 120 may face to the left, resulting in a connection wherein crown 108b is rotatably disposed within ring 112′ and crown 106a is fused to wall 116′ so as to block slot 120. In another embodiment that will be discussed in further detail below and is illustrated in FIGS. 28-32, slot 120 faces to the left, resulting in a connection 310 where crown 106a is rotatably and removably disposed within ring 112′ and crown 108b is fused to the outside of wall 116′ opposite or away from slot 120 such that slot 120 remains open. In yet another embodiment that will also be discussed in further detail below, rings 112′ may be oriented such that slot 120 is disposed facing radially away from mandrel 130. Rings 112′ may be temporarily mounted on mandrel 130 in any direction or orientation as long as slots 120 are unobstructed by mandrel 130.

As waveform 124 is helically wrapped around mandrel 130, a crown 106a of waveform 124 becomes aligned with slot 120 of a ring 112′, as shown in FIG. 12. Crown 106a is then passed through slot 120 to be received within lumen 118′ of ring 112′, as shown in FIG. 13. Although not required in all embodiments of the disclosure using ring 112′, in the illustrated embodiment crown 106a is thicker or greater in diameter than the width of slot 120 such that inserting crown 106a through slot 120 requires sufficient force to momentarily spread slot 120. Thus, insertion of crown 106a into lumen 118′ is a snap-fit. In the embodiment shown, as waveform 124 is helically wrapped around mandrel 130, the crowns 106a are disposed on the right side of rings 112′ and then moved towards the left to enter lumens 118′ through slots 120.

As waveform 124 continues to be helically wrapped around mandrel 130, a crown 108b of the adjacent band spans slot 120 and abuts the edges thereof, but crown 108b remains outside of lumen 118′ of ring 112′, as shown in FIG. 14. This process is repeated as waveform 124 is helically wrapped around mandrel 130 until all of the selected crowns 106a are rotatably disposed in lumens 118′ of rings 112′ and the selected crowns 108b are disposed spanning the outside of slots 120 of rings 112′. As would be understood by those of ordinary skill in the art, a number of rings 112′ may be located along mandrel 130 so as to provide rotatable crown-to-crown connections of the quantity and pattern desired.

When the waveform 124 has been completely wrapped around mandrel 130, at least one of the edges of slot 120 of ring 112′ is attached to crown 108b by fusion 114′, as shown in FIGS. 15 and 16. In the joint thus formed, crown 108b permanently spans or blocks slot 120 to substantially close the circumference of ring 112′ such that crown 106a is securely enclosed within lumen 118′ while still being free to rotate therewithin. The adhesive 132 or other connection between rings 112′ and mandrel 130 may then be removed or otherwise disrupted, leaving stent 100 free to be slid off of mandrel 130. Further processing steps such as cleaning, polishing, etc. known to those of ordinary skill in the art may then be performed to finish stent 100.

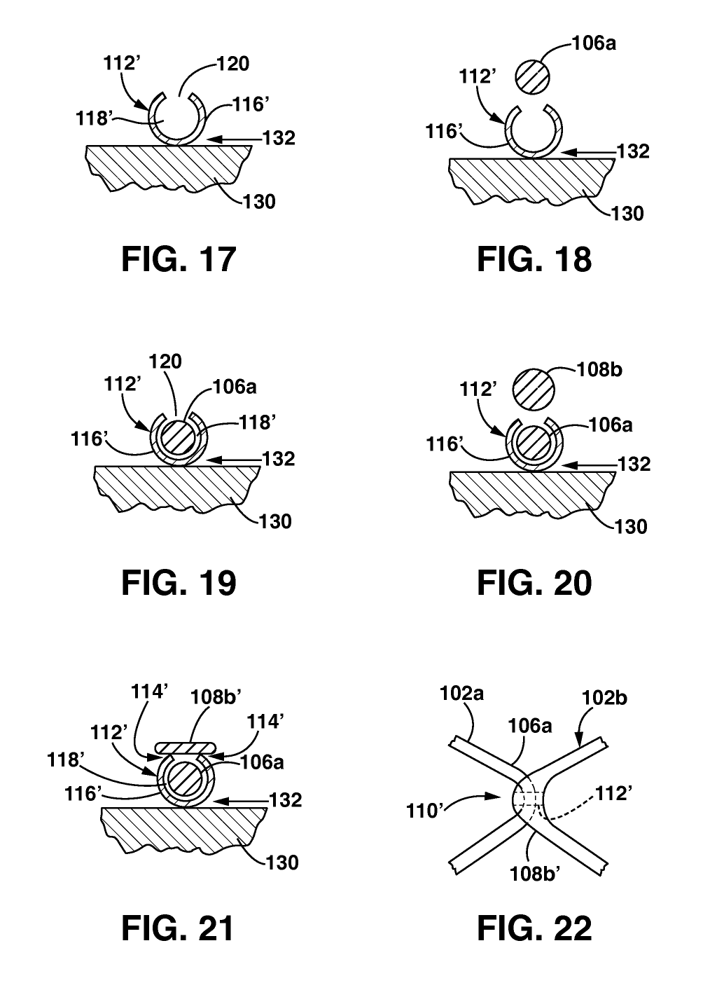

FIGS. 17-22 illustrate an alternative embodiment of stent 100 and a method of making the stent using rings 112′. As described above and illustrated in FIGS. 10-16, C-shaped rings 112′ may be temporarily affixed to an outer surface of mandrel 130 at appropriate locations to receive selected crowns 106a as waveform 124 is helically wrapped around mandrel 130. In the current embodiment, however, rings 112′ are oriented such that slot 120 is disposed facing radially away from mandrel 130 as shown in FIG. 17. As waveform 124 is helically wrapped around mandrel 130, a crown 106a of waveform 124 becomes aligned with slot 120 of a ring 112′, as shown in FIG. 18. Crown 106a is then moved through slot 120 to be received within lumen 118′ of ring 112′, as shown in FIG. 19. As waveform 124 continues to be helically wrapped around mandrel 130, a crown 108b of the adjacent band spans slot 120 and abuts the outer edges thereof, but crown 108b remains outside of lumen 118′ of ring 112′, as shown in FIG. 20.

When the waveform 124 has been completely wrapped around mandrel 130, one or more edges of slot 120 of ring 112′ are attached to crown 108b by fusion 114′. In the joint thus formed, crown 108b spans and blocks slot 120 such that crown 106a is securely enclosed within lumen 118′ while still being free to rotate therewithin. After the fusion step, the assembly shown in FIG. 20 resembles the assembly shown in FIG. 15 except that crown 108b is disposed above or radially overlapping ring 112′ and crown 106a. Such an arrangement defines increased stent wall thickness in the overlapped crown-to-crown connections 110, and a stent having such connections 110 that are approximately twice the radial thickness of the remainder of the stent may be suitable for some clinical applications. However, it may be desirable to reduce this overlapped joint thickness by, in one example, flattening crown 108b′ as shown in FIGS. 21 and 22. In preparation for making such connections 110′, one or more crowns 108b′ may be flattened to reduce their thickness after waveform 124 is formed but before it is helically wrapped around mandrel 130. As shown in FIGS. 21 and 22, a crown 108b′ that is flattened to reduce its thickness will tend to have an increased width unless other material removal steps are taken, and such steps are contemplated though not illustrated herein.

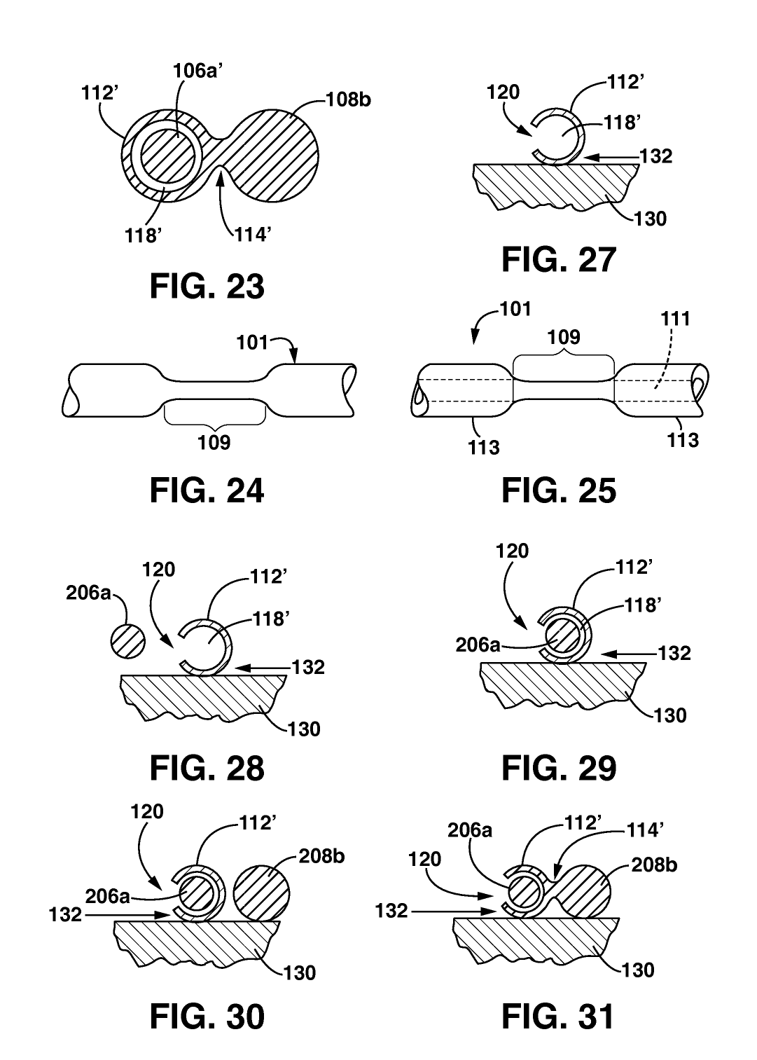

Another embodiment of the disclosure provides an alternative or additional means of reducing the radial thickness of crown-to-crown connections 110 incorporating ring 112′. In the embodiments shown in FIGS. 11-22 ring 112′ is sized and shaped to rotatably surround the portion of wire 101 that forms crown 106a. The result is that the outside diameter or radial thickness of ring 112′ is larger than the radial thickness of adjacent crown 108b except in the examples shown in FIGS. 21 and 22 where crown 108b′ has been flattened. As illustrated in FIG. 23, crown 106a′ is modified to have a reduced diameter as compared to the diameter of crown 106a such that ring 112′ need be no larger in diameter than unflattened crown 108b and still provide a lumen 118′ that rotatably receives crown 106a′. As illustrated in FIGS. 27 and 28, material may be selectively removed from wire 101 to provide a reduced diameter in one or more portions 109 having sufficient length to form crowns 106a′ when wire 101 is bent into waveform 124. FIG. 24 illustrates an embodiment wherein wire 101 comprises a single material and portion 109 may be reduced in diameter by such techniques as centerless grinding, chemical etching or other suitable methods of removing material. FIG. 25 illustrates an embodiment wherein wire 101 comprises a composite material such as a drawn filed tube (DFT) having a core of a first material 111 surrounded by a layer of a second material 113. First material 111 may have a substantially higher melt temperature than second material 113 such that portion 109 may be reduced in diameter by such techniques as selective vaporization of material 113 using a laser. In a non-limiting example, first core material 111 may be nickel-cobalt-chromium-molybdenum “superalloy” and second layer material 113 may be tantalum, tungsten, molybdenum, columbium (niobium), iridium, osmium, or zirconium.

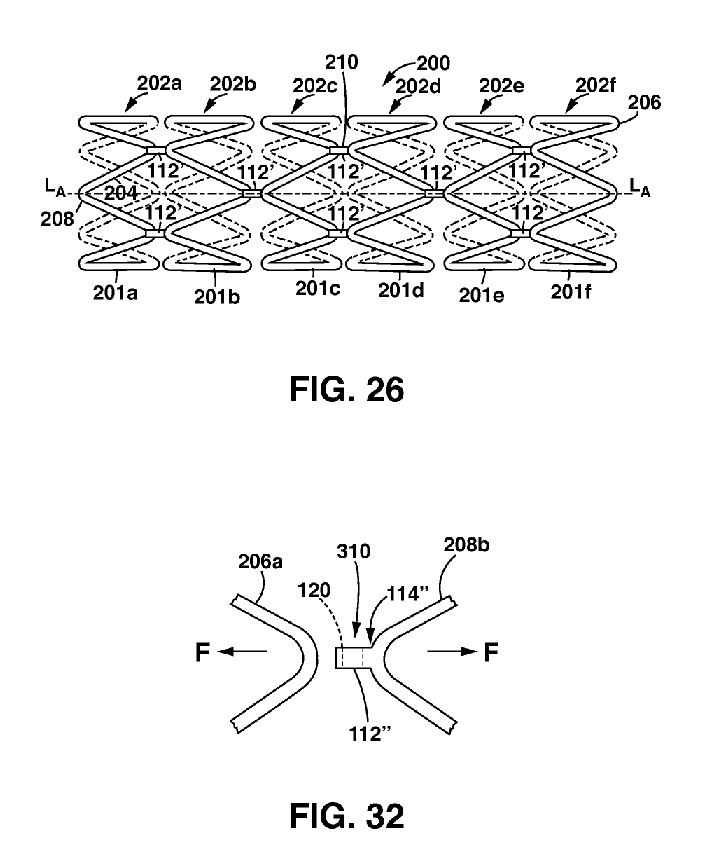

Another embodiment of a flexible stent 200 is shown in FIG. 26. Instead of the helically wound stent 100 of FIG. 1, stent 200 is formed from separate cylindrical elements 202a-202f disposed adjacent to each other and connected together along a common longitudinal axis L_{A} at connections 210. Those of ordinary skill in the art would recognize that more or fewer cylindrical elements 202 may be connected to form the stent 200.

In this particular embodiment, each cylindrical element 202 is formed from meandering, zigzag or generally sinusoidally shaped segments including generally straight struts 204 interconnected by crowns 206, 208. As is done above with respect to stent 100, crowns opening to the right have been arbitrarily labeled with reference numeral 208 and crowns opening to the left have been arbitrarily labeled with reference numeral 206. Cylindrical elements 202 may be identical to or similar to cylindrical elements found in the DRIVER® coronary stent, currently available from the assignee of the present invention, Medtronic CardioVascular, Inc. The invention hereof is not limited to the pattern shown in FIG. 26. Cylindrical elements 202 may be formed from a deformed ring or wire-like structure 201, as described in U.S. Pat. No. 6,344,053 to Boneau, or elements 202 may be cut or etched from a tube, for example, or may be formed from any other method known to those of ordinary skill in the art.

Adjacent cylindrical elements 202 are connected together by generally C-shaped rings 112′. Rings 112′ are used to make stent 200 because cylindrical elements 202 are each generally closed, lacking free ends for receiving rings 112 threaded thereon. However, those of ordinary skill in the art would recognize that cylindrical elements 202 can be made with free ends that may be butt welded to close the cylindrical element after rings 112 have been threaded thereon.

The cylindrical elements 202 are located adjacent to each other. For example, and not by way of limitation, cylindrical elements may be located adjacent to each other on a temporary mandrel or a rod. Connections 210 may be formed by the series of steps described above for making connections 110 using rings 112′ as illustrated in FIGS. 10-16. Generally C-shaped rings 112′ may be temporarily affixed to an outer surface of mandrel 130 at appropriate locations to receive selected crowns 106a as cylindrical elements 202 are slid onto mandrel 130, as shown in phantom in FIG. 10. Rings 112′ may be temporarily affixed to mandrel 130 by an adhesive 132, or any other attachment means that can be selectively removed as described below. FIG. 11 shows a cross-section through a portion of mandrel 130 where a ring 112′ is located. Those of ordinary skill in the art would recognize that the orientation of ring 112′ on mandrel 130 may be varied as described above regarding the method of making stent 100.

As cylindrical element 202 is slid onto mandrel 130, a crown 106a of element 202 becomes aligned with slot 120 of a ring 112′, as shown in FIG. 12. Crown 106a is then moved through slot 120 to be received within lumen 118′ of ring 112′, as shown in FIG. 13. Although not required in all embodiments of the disclosure using ring 112′, in the illustrated embodiment crown 106a is thicker or greater in diameter than the width of slot 120 such that inserting crown 106a through slot 120 requires sufficient force to momentarily spread slot 120. Thus, insertion of crown 106a into lumen 118′ is a snap-fit. In the embodiment shown, as cylindrical element 202 is slid onto mandrel 130, the crowns 106a are disposed on the right side of rings 112′ and then moved towards the left to enter lumens 118′ through slots 120.

As cylindrical element 202 is slid onto mandrel 130, a crown 108b of the adjacent band spans and abuts the outside edges of slot 120 of ring 112′, but crown 108b remains outside of lumen 118′ of ring 112′, as shown in FIG. 14. This process is repeated as cylindrical elements 202 are sequentially loaded onto mandrel 130 until all of the selected crowns 106a are rotatably disposed in lumens 118′ of rings 112′ and the selected crowns 108b are disposed spanning slots 120 of rings 112′ on the outside of rings 112′. As would be understood by those of ordinary skill in the art, a number of rings 112′ may be located along mandrel 130 so as to provide rotatable crown-to-crown connections 210 of the quantity and pattern desired.

When all of the desired cylindrical elements 202 have been mounted on mandrel 130, at least one of the outer edges of slot 120 is attached to crown 108b by fusion 114′, as shown in FIGS. 15 and 16. In the joint thus formed between ring 112′, crown 108b permanently blocks slot 120 and substantially closes the circumference of ring 112′ such that crown 106a is securely enclosed within lumen 118′ while still being free to rotate therewithin. The adhesive 132 or other connection between rings 112′ and mandrel 130 may then be removed or otherwise disrupted, leaving stent 200 free to be slid off of mandrel 130. Further processing steps such as cleaning, polishing, etc. known to those of ordinary skill in the art may then be performed to finish stent 200.

FIGS. 27-32 illustrate an alternative embodiment and method of making stent 200 with one or more rotatable and releasable connections 310. FIG. 32 illustrates a connection 310 incorporating ring 112′ to join crown 206a and crown 208b in a snap-fit that may be selectively disconnected, released or un-snapped by longitudinal separation forces F. Connections 310 may be disconnected in-vivo to deploy and implant fewer than all of the cylindrical elements 202 comprising stent 200. One or more cylindrical elements 202 can be deployed at a single treatment site while still connected to each other. Alternatively, working from one end of stent 200, one or more selected cylindrical elements 202 may be disconnected from other elements 202 and distributed to different vascular target locations. U.S. Patent Application Publication No. US2003/0135266A1 to Chew et al., which is incorporated herein by reference in its entirety, teaches a stent deployment system having a plurality of cylindrical prostheses carried in an end-to-end manner and being connected by various types of linkages that may be separated either with or without material breakage.

FIGS. 27-31 illustrate a method of making connections 310 that may be snapped-fitted together or snapped apart. FIG. 27 shows a cross-section through a portion of mandrel 130 where ring 112′ is temporarily affixed to mandrel 130 by an adhesive 132 with slot 120 facing left. As a cylindrical element 202 is slid onto mandrel 130, a crown 206a of cylindrical element 202 becomes aligned with slot 120 on the left of a ring 112′, as shown in FIG. 28. Crown 206a is then moved through slot 120 to be rotatably received within lumen 118′ of ring 112′, as shown in FIG. 29. As described in an above embodiment, crown 206a is thicker or greater in diameter than slot 120 such that inserting crown 206a through slot 120 requires sufficient force to momentarily expand ring 112′ thus spreading slot 120. Once crown 206a is disposed within lumen 118′, ring 112′ including slot 120 elastically recovers substantially to its original dimensions. Thus, insertion of crown 206a into lumen 118′ is a snap-fit. As the next cylindrical element 202 is slid onto mandrel 130, a crown 208b abuts the right side of ring 112′ as shown in FIG. 30.

This process is repeated as the cylindrical elements 202 are loaded onto mandrel 130, such that all of the selected crowns 206a are rotatably and removably snap-fitted into lumens 118′ of rings 112′ and the selected crowns 208b abut rings 112′ on the outer surface of rings 112′ without spanning or obstructing slot 120. As would be understood by those of ordinary skill in the art, a number of rings 112′ may be located along mandrel 130 so as to provide rotatable and selectively disconnectable crown-to-crown connections 310 of the quantity and pattern desired. Then, the outside surface of wall 116′ opposite or otherwise away from slot 120 of ring 112′ is attached to crown 208b by fusion 114′, as shown in FIG. 31. As would be understood by one of skill in the art, rotatable and disconnectable connection 310 may comprise ring 112′ disposed in one of various positions such that slot 120 is oriented in a range of directions from left-facing to radially away from the mandrel to right-facing, as long as slot 120 is not obstructed by either mandrel 130 or fused crown 208b.

In connections 310 thus formed, fusion 114′ permanently attaches ring 112′ to crown 208b. Crown 206a is free to rotate within lumen 118′ and is capable of being removed from lumen 118′ through open slot 120 by reversing the snap-fit. The adhesive 132 or other connection between rings 112′ and mandrel 130 may be removed or otherwise disrupted, leaving stent 200 free to be slid off of mandrel 130. Further processing steps such as cleaning, polishing, etc. known to those of ordinary skill in the art may then be performed to finish stent 200.

Those of ordinary skill in the art would recognize that flexible connections 110, 210, 310 may be combined with other known types of connections in a single stent to achieve a variation in flexibility along the length of the stent. For example, and not by way of limitation, the cylindrical elements in the center of the stent may have connections 110, 210, 310 described herein, and the cylindrical elements near the ends of the stent may have the crowns directly welded to each other, as is known in the art, or vice versa.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of making a tubular stent comprising the steps of:
aligning a first cylindrical element and a second cylindrical element adjacent to each other along a common longitudinal axis, wherein the first and second cylindrical elements each include a wire in a zigzag configuration defined by a series of generally straight segments interconnected by crowns;
mounting at least one connecting ring on the first cylindrical element such that the wire of the first cylindrical element is rotatably disposed through a lumen of the connecting ring and the wire of the second cylindrical element is disposed abutting an exterior surface of the connecting ring; and
fusing the exterior surface of the connecting ring to the wire of the second cylindrical element to rotatably connect the first cylindrical element to the second cylindrical element.

2. The method of claim 1, wherein the mounting step further comprises:
locating the at least one connecting ring at a selected crown.

3. The method of claim 2 further comprising:
attaching the at least one connecting ring temporarily to the wire after the locating step; and
removing the temporary attachment between the at least one ring and the wire after the fusing step.

4. The method of claim 3, wherein the temporary attachment is adhesively bonding the connecting ring to the wire.

5. The method of claim 1, wherein the at least one connecting ring has an open longitudinal slot to define a generally C-shaped ring and the mounting step further comprises:
sliding the wire of the first cylindrical element transversely through the slot into the lumen of the connecting ring.

6. The method of claim 5 further comprising:
affixing a plurality of the C-shaped connecting rings along a cylindrical mandrel and wherein the aligning step further comprises:
sliding the first and second cylindrical elements onto the mandrel.

7. The method of claim 6 further comprising:
adhesively affixing the connecting rings to the mandrel.

8. The method of claim 7 further comprising:
removing the adhesive such that the connecting rings are detached from the mandrel.

9. The method of claim 5, wherein the open slot forms a pair of opposing edges where it intersects the exterior surface of the connecting ring and wherein the fusing step further comprises:
fusing at least one of the edges to a crown of the second cylindrical element such that the crown of the second cylindrical element blocks the slot sufficiently to retain the crown of the first cylindrical element rotatably disposed within the lumen of the connecting ring.

10. The method of claim 5 further comprising, before the mounting step, selectively reducing a diameter or thickness of the wire in the regions forming one or more crowns.

11. The method of claim 5, wherein the open slot has a width that is narrower than a thickness of the wire such that the sliding step further comprises:
snap-fitting the wire of the first cylindrical element transversely through the slot into the lumen of the connecting ring; and
wherein the snap-fit is reversible.

12. A flexible tubular stent comprising:
a plurality of cylindrical bands aligned generally along a common longitudinal axis, wherein the plurality of bands includes at least a first band having a plurality of first crowns and a second band adjacent to the first band and having a plurality of second crowns; and
a ring connecting one of the first crowns to one of the second crowns, wherein the first crown is rotatably disposed through a lumen of the ring and the second crown is fused to a wall of the ring and is disposed outside of the lumen of the ring.

13. The flexible stent of claim 12, wherein the cylindrical elements comprise a wire formed into a zigzag shape.

14. The stent of claim 13 wherein the connecting ring is generally C-shaped as defined by an open longitudinal slot having edges where the slot intersects an outer surface of the ring and wherein the second crown is disposed spanning the slot and is fused to at least one of the slot edges such that the second crown blocks the slot to retain the first crown rotatably disposed within the lumen of the connecting ring.

15. The stent of claim 14 wherein the first crown rotatably disposed within the lumen of the C-shaped connecting ring has a diameter that is less than the diameter of the wire comprising the second crown.

16. The stent of claim 15 wherein an outer diameter of the connecting ring is substantially equal to a diameter of the wire comprising the second crown.

17. The stent of claim 13 wherein the connecting ring is generally C-shaped as defined by an open longitudinal slot and wherein the fused second crown is disposed so as to not block the slot and wherein the open slot has a width that is narrower than a thickness of the wire forming the first crown such that the first crown may be reversibly snap-fitted transversely through the slot into the lumen of the ring to retain the first crown rotatably disposed therewithin.
